(19) United States
(12) Patent Application Publication
(10) Pub. No.: US 2026/0053587 A1
(43) Pub. Date: Feb. 26, 2026

(54) METHOD AND APPARATUS FOR TENSIONING A CONTROL WIRE OF A MEDICAL DEVICE

(71) Applicant: TANGENT ENDOSCOPY, LLC, Camden, DE (US)

(72) Inventors: Scott Richard ARP, Miami, FL (US); Joseph Paul AGUILA, Fort Lauderdale, FL (US); Berry LAMY, Boynton Beach, FL (US); Angel MENDOZA, Tamarac, FL (US)

(21) Appl. No.: 18/991,591

(22) Filed: Dec. 22, 2024

Related U.S. Application Data
(60) Provisional application No. 63/686,060, filed on Aug. 22, 2024.

(51) Int. Cl. A61B 34/00; A61B 1/005; A61M 25/01

(52) U.S. Cl. CPC A61B 34/71; A61B 1/0057; A61B 2034/715; A61M 25/0147

(57) ABSTRACT

A method and apparatus for tensioning a control wire in a medical device, wherein the apparatus comprises a lug adapted to be fixedly attached to a housing of the medical device; a wire guide defining an aperture through which the control wire extends; and a cantilever spring having a first end coupled to the lug and a second end coupled to the wire guide for applying force to the control wire. The apparatus is used in a medical device such as an endoscope.

### RELATED APPLICATION

This application claims benefit to U.S. Provisional Patent Application Ser. No. 63/686,060 filed 22 Aug. 2024 entitled “Method and Apparatus for Tensioning a Control Wire of a Medical Device,” which is hereby incorporated herein by reference in its entirety.

### BACKGROUND

### Field

Embodiments of the present invention generally relate to medical devices and, in particular, to a method and apparatus for tensioning a control wire of a medical device.

### Description of the Related Art

A typical medical device such as an endoscope comprises an actuator and a substantially hollow, flexible shaft extending from the actuator. The actuator manipulates at least one control wire that extends, along the inside of the shaft, from the actuator to a distal end of the shaft. The actuator generally comprises a take-up wheel coupled to a control knob or slider. The at least one control wire is attached to the take-up wheel. As the knob or slider is manually moved, the take-up wheel rotates and more or less control wire wraps around the wheel. As the control wire is taken-up by the wheel, the distal end of the flexible shaft is curved in a specific direction depending on the attachment point of the distal end of the wire to the flexible shaft.

If the medical device comprises two wires, the wires are attached to opposite sides of the wheel and manipulation of the actuator moves the distal end of the shaft left or right, or up or down. If the medical device comprises four wires, the wires are attached to two independently controllable take-up wheels and manipulation of the actuator moves the distal end of the shaft left, right, up or down, i.e., three-dimensional manipulation.

As the actuator is manipulated, the wire that is tensioned by the take-up wheel moves the distal end in a particular direction. The wires that are not tensioned will have slack in them. Upon changing direction of the manipulator, the slack must be taken-up before the distal end of the shaft will move. Such slack causes a lack of end motion for a time period until the slack is taken-up. This slack induced hysteresis can result in inaccurate positioning of the endoscope distal end and incorrect positioning of a medical instrument located at the distal end.

Therefore, there is a need for a method and apparatus for tensioning a control wire of a medical device.

### SUMMARY

A method and apparatus for tensioning a control wire of a medical device is provided substantially as shown in and/or described in connection with at least one of the figures, as set forth more completely in the claims.

Various features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the above recited features of the present invention can be understood in detail, a particular description of the invention, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a perspective view of an medical device in accordance with at least one embodiment of the invention;

FIG. 2 depicts a cross-sectional view of an actuator of the medical device of FIG. 1 in accordance with at least one embodiment of the invention;

FIG. 3 depicts a housing removed, first side view of the actuator of FIG. 2 in accordance with at least one embodiment of the invention;

FIG. 4 depicts a housing removed, second side view of the actuator of FIG. 2 in accordance with at least one embodiment of the invention;

FIG. 5 depicts top plan view of a control wire tensioner in accordance with at least one embodiment of the invention;

FIG. 6 depicts side view of a control wire tensioner of FIG. 5 in accordance with at least one embodiment of the invention;

FIG. 7 depicts a side cross-sectional view of the shaft tip in accordance with at least one embodiment of the invention; and

FIG. 8 depicts an end cross-sectional view of the shaft tip along lines 8-8 in FIG. 7 in accordance with at least one embodiment of the invention.

### DETAILED DESCRIPTION

Embodiments of the present invention include a method and apparatus for tensioning on at least one control wire of a medical device such as an endoscope. The medical device comprises an actuator coupled to a substantially hollow, flexible shaft. The actuator is coupled to at least one control wire that extends internal of the shaft from the actuator to a location near a distal end (or tip) of the shaft. Manipulation of the actuator pulls on the control wire and causes the distal end of the shaft to move in a specific direction. In one embodiment of the invention, the actuator comprises a tensioner to maintain continuous tension upon the at least one control wire. The tensioner improves the movement responsiveness of the distal end of the endoscope by continuously removing slack from the control wire. In a specific embodiment, the tensioner comprises a cantilever spring (i.e., a leaf spring) extending between a mounting lug and a wire guide. The mounting lug anchors the lug in a fixed position. As the wire moves through the wire guide, the spring applies tension on the wire via the wire guide. Consequently, the control wire is always under tension and immediately moves the distal end of the shaft whenever the actuator changes direction of the shaft tip.

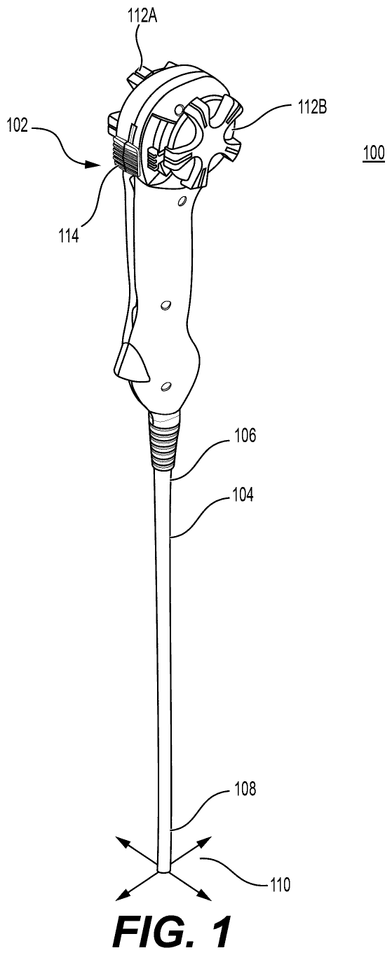

FIG. 1 depicts a perspective view of a medical device (e.g., endoscope 100) in accordance with at least one embodiment of the invention. The endoscope 100 comprises an actuator 102 coupled to a hollow flexible shaft 104. The shaft 104 comprises a proximal end 106 coupled to the actuator 102 and a distal end 108 (also referred to as a tip) that is directionally manipulated by the actuator 102 (as indicated by arrows 110). In one embodiment, the actuator 102 comprises at least one knob 112 (shown as two knobs 112A and 112B) and/or sliders 114 that are manually manipulated to move the distal end 108 of the shaft 104. In other embodiments, the actuator 102 comprises automated manipulators such as stepper motors, solenoids, and the like (not shown) that can be electronically manipulated to move the distal end 108 of the shaft 104.

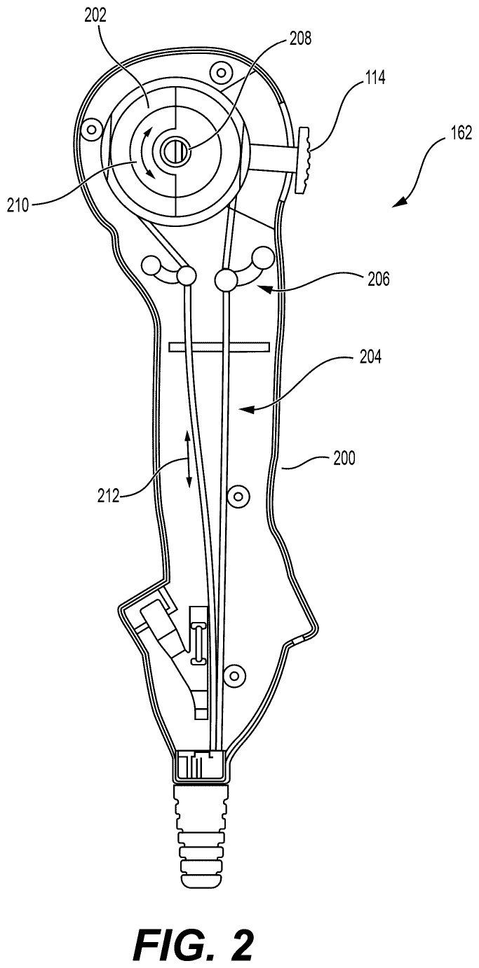

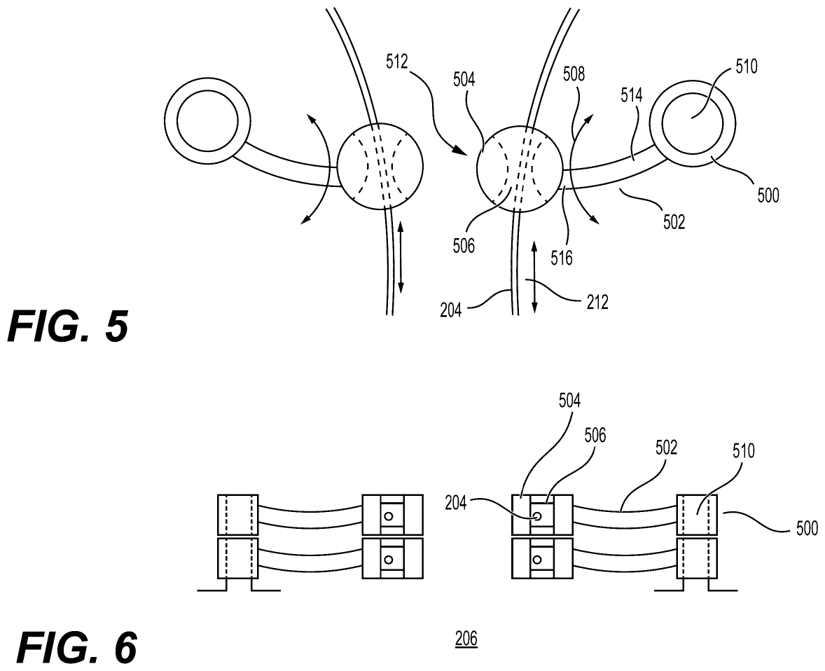

FIG. 2 depicts side cross-sectional view of the actuator 102 of the endoscope 100 of FIG. 1 in accordance with at least one embodiment of the invention. The actuator 102 comprises a housing 200 that is shaped to be held in a medical professional's hand while the knob (not shown in FIG. 2) and/or slider 114 are manipulated. The actuator 102 comprises at least one take-up wheel 202 that is manipulated by either a knob, slider or electronic manipulators. At least one control wire 204 is coupled to at least one take-up wheel 202. A tensioner 206 is positioned along the at least one control wire 204 to continuously apply tension to the at least one control wire. A detailed description of the tensioner 206 structure and operation is provided with respect to FIGS. 5 and 6 below.

The take-up wheel 202 rotates about an axis 208 (arrow 210). The at least one control wire 204 is attached to the perimeter of the wheel 202. As the wheel 202 is rotated, the at least one control wire 204 linearly moves (arrow 212). In an embodiment with two control wires, the wires are attached to the take-up wheel on opposite sides of the perimeter of the wheel. Rotation of the wheel causes the tip to move left-right or up-down depending on the location of the attachment point of the wire near the tip. In an embodiment where the tip may move in three dimensions, four wires are used with two wires coupled to two separate and independently rotatable take-up wheels. The take-up wheels are stacked adjacent each other and independently rotate about the same axis.

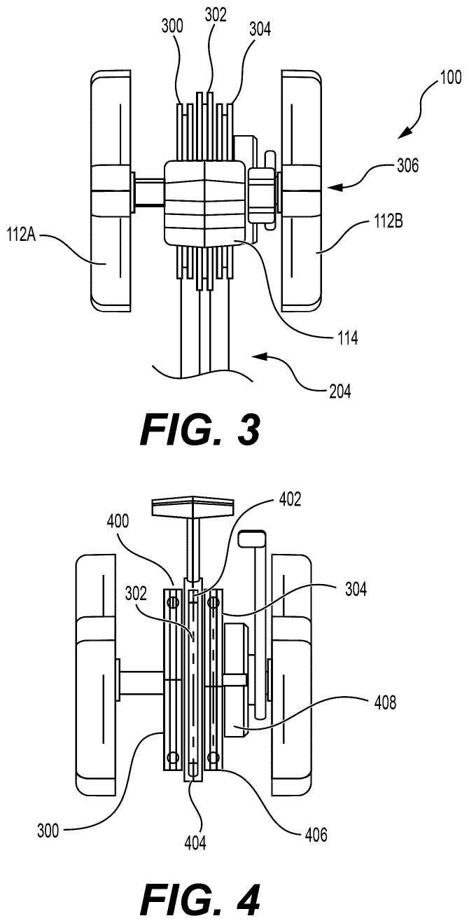

FIG. 3 depicts a housing removed, first side view of the actuator 100 of FIG. 2 in accordance with at least one embodiment of the invention. In an embodiment for three-dimensional tip movement, the actuator comprises three stacked take-up wheels 300, 302, 304 having a common rotational axis 306 to facilitate independent manipulation of each control wire 204. In the depicted embodiment, wheel 302 is manipulated by the slider 114, while wheels 300 and 304 are manipulated by knobs 112A and 112B. In some embodiments, the knobs 112A and 112B are on a common axle with the wheels 300 and 304 such that both knobs turn the wheels simultaneously. In other embodiments, the knobs and wheels may operate independently. As each wheel 300, 302, 304 is rotated, the control wires 204 move linearly to move the distal end of the shaft left, right, up and/or down.

FIG. 3 depicts a housing removed, second side view of the actuator 100 of FIG. 2 in accordance with at least one embodiment of the invention. The attachment points 400, 402, 404 and 406 for the wires 204 on the wheels 300, 302, and 304 are shown. Additionally, the actuator 100 comprises a brake mechanism 408 that is used to lock the wheels 300, 302 and 304 in position to maintain the orientation of the distal end of the shaft.

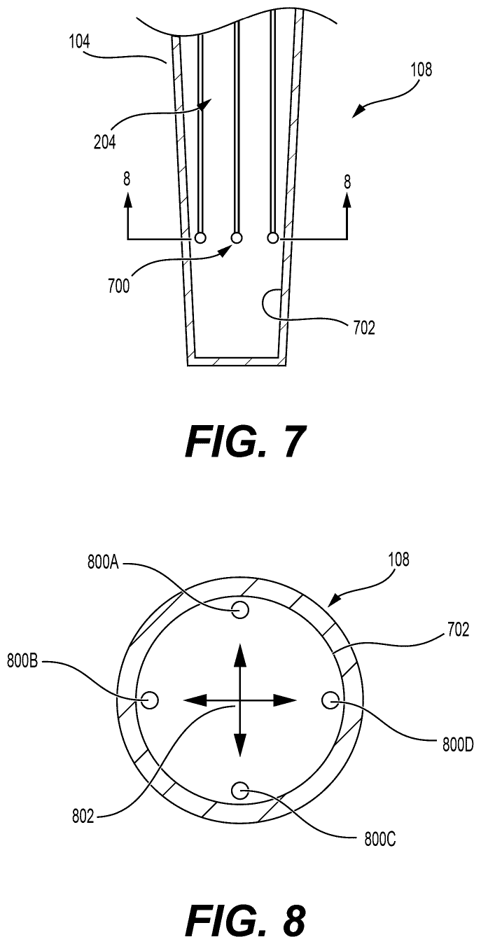

FIG. 7 depicts a side cross-sectional view of the endoscope shaft distal end 108 in accordance with at least one embodiment of the invention. FIG. 8 depicts an end cross-sectional view along lines 8-8 in FIG. 7 of the endoscope shaft distal end 108 in accordance with at least one embodiment of the invention. The distal end 108 forms the tip of the hollow, flexible shaft 104 in FIG. 1. Within the shaft, the ends 700 of the control wires 204 are attached to the inside surface 702 of the shaft 104 proximate the distal end 108. As shown in FIG. 8, for a four-wire endoscope, the wires 204 are attached at 90 degree locations 800A, 800B, 800C, and 800D around the circumference of the inner surface 702 of the shaft. Linear motion of the wires 204 causes the tip to be moved in a particular direction represented by the arrows 802.

FIG. 5 depicts top plan view of the control wire tensioner 206 in accordance with at least one embodiment of the invention. FIG. 6 depicts side view of the control wire tensioner 206 in accordance with at least one embodiment of the invention. The tensioner 206 comprises one tensioner element 512 for each control wire 204. As such, for an embodiment with four control wires 204, there are four tensioner elements 512. The tensioner element 512 comprises a lug 500, a cantilever spring 502 (sometimes referred to as a leaf spring) and a wire guide 504. The lug 500 is anchored in a fixed position and the cantilever spring 502 extends between the lug 500 and the wire guide 504, i.e. a first end 514 of the cantilever spring 502 is attached to the lug 500 and a second end 516 of the cantilever spring 502 is attached to the wire guide 504. The wire guide 504 comprises an aperture 506 through the guide 504. The control wire 204 is thread through the aperture 506. In one embodiment, the aperture 506 has a pair of arcuate side walls (surfaces). The aperture is defined by the pair of arcuate surfaces that face each other. The lug 500 may be anchored in any way. In one exemplary embodiment, the lug 500 defines a substantially cylindrical aperture that is attached to a stud 510 extending from the actuator housing 200 in FIG. 2. The lug 500 may be press fit onto the stud 510 or affixed with at least one of adhesive, a pin, a key way, or any other affixation technique that retains the lug in a fixed position.

In operation, as the wire 204 moves (arrow 212) through the wire guide 504, the cantilever spring 502 is compressed to apply force to the wire guide 504 to tension the control wire 204. The tension ensures that the wire does not have any slack that would delay responsiveness of the shaft movement.

Here multiple examples have been given to illustrate various features and are not intended to be so limiting. Any one or more of the features may not be limited to the particular examples presented herein, regardless of any order, combination, or connections described. In fact, it should be understood that any combination of the features and/or elements described by way of example above are contemplated, including any variation or modification which is not enumerated, but capable of achieving the same. Unless otherwise stated, any one or more of the features may be combined in any order.

As above, figures are presented herein for illustrative purposes and are not meant to impose any structural limitations, unless otherwise specified. Various modifications to any of the structures shown in the figures are contemplated to be within the scope of the invention presented herein. The invention is not intended to be limited to any scope of claim language.

Where conditional language is used, including, but not limited to, “can,” “could,” “may” or “might,” it should be understood that the associated features or elements are not required. As such, where conditional language is used, the elements and/or features should be understood as being optionally present in at least some examples, and not necessarily conditioned upon anything, unless otherwise specified.

Where lists are enumerated in the alternative or conjunctive (e.g., one or more of A, B, and/or C), unless stated otherwise, it is understood to include one or more of each element, including any one or more combinations of any number of the enumerated elements (e.g. A, AB, AC, ABC, ABB, etc.). When “and/or” is used, it should be understood that the elements may be joined in the alternative or conjunctive.

While the foregoing is directed to embodiments of the present invention, other and further embodiments of the invention may be devised without departing from the basic scope thereof, and the scope thereof is determined by the claims that follow.

1. Apparatus for tensioning a control wire of a medical device comprising:
a lug adapted to be fixedly attached to a housing of the medical device;
a wire guide defining an aperture through which the control wire extends; and
a cantilever spring having a first end coupled to the lug and a second end coupled to the wire guide.

2. The apparatus of claim 1 wherein the aperture of the wire guide comprises a pair of arcuate surfaces which face each other and define the aperture through which the control wire extends.

3. The apparatus of claim 1 wherein the lug defines a substantially cylindrical aperture.

4. The apparatus of claim 3 wherein the substantially cylindrical aperture is adapted to mount to a stud extending from the housing.

5. The apparatus of claim 4 wherein the substantially cylindrical aperture is affixed to the stud by at least one of press fit, adhesive, a pin, or a key way.

6. The apparatus of claim 1 wherein the cantilever spring maintains the control wire in tension.

7. The apparatus of claim 6 wherein the tension ensures the control wire does not become slack.

8. The apparatus of claim 1 further comprising a plurality of tensioner elements having a lug, cantilever spring and wire guide and wherein each tensioner element in the plurality of tensioner elements tensions a control wire.

9. A method of operation of a tensioner for tensioning a control wire of a medical device where the tensioner comprises a lug adapted to be fixedly attached to a housing of the medical device, a wire guide defining an aperture through which the control wire extends, and a leaf spring having a first end coupled to the lug and a second end coupled to the wire guide, the method comprising:
as the control wire moves through the wire guide aperture, the cantilever spring applies a force to the control wire to apply tension to the control wire.

9. (canceled)

10. A medical device comprising:
an actuator coupled to at least one guide wire;
a tensioner for applying force to the at least one guide wire to tension the at least one guide wire, where the tensioner comprises at least one tensioner element comprising:
a lug adapted to be fixedly attached to a housing of the medical device;
a wire guide defining an aperture through which a control wire of the at least one control wires extends; and
a cantilever spring having a first end coupled to the lug and a second end coupled to the wire guide.

11. The medical device of claim 10 wherein the aperture of the wire guide comprises a pair of arcuate surfaces which face each other and define the aperture through which the control wire extends.

12. The medical device of claim 10 wherein the lug defines a substantially cylindrical aperture.

13. The medical device of claim 12 wherein the substantially cylindrical aperture is adapted to mount to a stud.

14. The medical device of claim 13 wherein the substantially cylindrical aperture is affixed to the stud by at least one of press fit, adhesive, a pin, or a key way.

15. The medical device of claim 10 wherein the cantilever spring maintains the control wire in tension.

16. The medical device of claim 15 wherein the tension ensures the control wire does not become slack.

17. The medical device of claim 10 wherein the tensioner comprises a plurality of tensioner elements wherein each tensioner element in the plurality of tensioner elements tensions a control wire.

18. The medical device of claim 10 wherein the actuator is coupled to a hollow shaft and an end of the at least one control wire is attached proximate to a distal end of the hollow shaft.

19. The medical device of claim 18 wherein the actuator imparts movement of the at least one control wire to move the distal end of the shaft.

20. The medical device of claim 19 comprises four control wires threaded through four tensioner elements and each control wire is attached at 90 degree locations of a circumference of the shaft.
